(19) United States
(12) Patent Application Publication
(10) Pub. No.: US 2022/0401124 A1
(43) Pub. Date: Dec. 22, 2022

(54) NEEDLE GUIDING DEVICE, AND SET COMPRISING A PLURALITY OF ELEMENTS

(71) Applicant: INLINE-MED GMBH, Magdeburg (DE)

(72) Inventors: Juan Sebastián SÁNCHEZ LÓPEZ, Magdeburg (DE); Sinja LAGOTZKI, Magdeburg (DE)

(21) Appl. No.: 17/782,858

(22) PCT Filed: Dec. 3, 2020
(86) PCT No.: PCT/EP2020/084477

§ 371 Date: Jun. 6, 2022

(30) Foreign Application Priority Data
Dec. 6, 2019 (DE) 10 2019 133 421.7

(51) Int. Cl. A61B 17/34; A61B 34/10

(52) U.S. Cl. CPC A61B 17/3403; A61B 34/10; A61B 90/11

(57) ABSTRACT

The invention relates to a needle guiding device for guiding and positioning a needle-shaped device on a patient, wherein the needle guiding device has a base element and a needle guiding element on which the needle-shaped device can be guided along its longitudinal extent, wherein the angular orientation of the needle guiding element can be set in two independent angular degrees of freedom with respect to the base element. The needle guiding device has an operating element, by means of which the angular orientation of the needle guiding element in the two angular degrees of freedom can be fixed or such fixing can be released again. The needle guiding device also has, for each of the two settable angular degrees of freedom, an angle scale and an angle pointer associated with the relevant angle scale, such that the user can, on the basis of the position of the relevant angle pointer with respect to the associated angle scale, read off a currently set angular orientation of the needle guiding element in the two angular degrees of freedom.

The invention relates to a needle-guiding device for guiding and positioning a needle-shaped device on a patient, the needle-guiding device having a base element and a needle-guiding element on which the needle-shaped device is guidable along its longitudinal extent, the angular orientation of the needle-guiding element relative to the base element being adjustable in two mutually independent degrees of angular freedom. The invention further relates to a set comprising a plurality of elements for carrying out an examination and/or treatment of the patient.

In general, the invention relates to the field of medical devices that can be used for the examination and/or treatment of patients, for example in the context of a biopsy or pain therapy. These procedures are usually carried out manually, with imaging by ultrasound, computed tomography or magnetic resonance imaging. It is already known to assist the physician by use of a mechanical auxiliary device in the form of a needle-guiding device, which simplifies the alignment of the biopsy needle and maintains the alignment during the performance of the biopsy. Such a needle-guiding device is known, for example, from WO 2019/101862 A1.

The invention is based on the object of specifying further improved aids for examination and/or treatment of a patient when using a needle-shaped device.

This object is achieved by a needle-guiding device for guiding and positioning a needle-shaped device on a patient, the needle-guiding device having a base element and a needle-guiding element on which the needle-shaped device is guidable along its longitudinal extent, the angular orientation of the needle-guiding element relative to the base element being adjustable in two mutually independent degrees of angular freedom, with the following features:
a) the needle-guiding device has an operating element by which the angular orientation of the needle-guiding element can be fixed in the two degrees of angular freedom or such fixing can be released again,
b) the needle-guiding device has an angle scale for each of the two adjustable degrees of angular freedom, and an angle indicator assigned to the respective angle scale such that a currently set angular orientation of the needle-guiding element in the two degrees of angular freedom can be read off by the user on the basis of the position of the respective angle indicator in relation to the assigned angle scale.

The needle-guiding device according to the invention considerably simplifies an examination and/or treatment of a patient by means of a needle-shaped device, for example in a biopsy. The angular orientation of the needle-guiding element can be fixed in two degrees of angular freedom or released again by a single operating element. In this way, the needle-guiding device is suitable for one-handed operation, at least as regards the actuation of the operating element. In addition, the use of the needle-guiding device is simplified by the presence of respective angle scales and associated angle indictors for each of the two adjustable degrees of angular freedom. In this way, the user can make the required angle adjustments quickly, and without additional accessories, by observing the angle indicators and angle scales.

The needle-guiding device can advantageously be designed in such a way that, with each setting of the angular orientation of the needle-guiding element in the two degrees of angular freedom, a needle-shaped device guided by the needle-guiding element always comes into contact with the patient's body at the same position, i.e., in the case of a biopsy, the biopsy needle always hits the same puncture point. The base element serves here to fasten the needle-guiding device to the patient's body, for example by means of a releasable adhesive. The needle-shaped device can be, for example, a biopsy needle, an injection needle or an ablation needle.

According to an advantageous embodiment of the invention, provision is made that the needle-guiding device has a clamping mechanism that is actuatable by the operating element, the angular orientation of the needle-guiding element relative to the base element in the two degrees of angular freedom being able to be fixed by clamping, through operation of the clamping mechanism by means of the operating element. Such a clamping mechanism can be realized in a comparatively simple manner, for example with threaded elements which can be tightened against each other and by means of which a part of the needle-guiding device is braced, thus allowing the angular orientation of the needle-guiding element to be fixed by clamping. With a clamping mechanism, it is possible in particular to obtain a stepless adjustment of the angular orientation of the needle-guiding element relative to the base element in the two degrees of angular freedom. In this way, a latching action can be avoided, such that in principle any desired angular orientations are set in the possible pivoting range of the needle-guiding element. The adjustment of the angular orientation of the needle-guiding element can also take place in a stepped manner in at least one degree of angular freedom or both degrees of angular freedom, for example via latch elements.

According to an advantageous embodiment of the invention, provision is made that the angular orientation of the needle-guiding element relative to the base element is steplessly adjustable in the two degrees of angular freedom. In this way, practically any setting of the angular orientation in both degrees of angular freedom is possible, such that, in terms of adjustment possibilities, the user is not limited by the design of the needle-guiding device.

According to an advantageous embodiment of the invention, provision is made that the needle-guiding device has a retaining bracket to which the needle-guiding element is fastened, wherein the retaining bracket in a first of the two degrees of angular freedom is movable with respect to the base element, and the needle-guiding element in a second of the two degrees of angular freedom is movable with respect to the retaining bracket. This permits simple and robust construction of the needle-guiding device. The retaining bracket can, for example, be formed in an arch shape and, by virtue of its arch shape, can support an arcuate mobility of the needle-guiding element with respect to the retaining bracket. The needle-guiding element can, for example, be guided in the manner of a carriage or by means of a retaining carriage on the retaining bracket and is displaceable along the retaining bracket in an arcuate movement corresponding to the arch shape of the retaining bracket and in this way is movable relative to the retaining bracket. The retaining bracket can for example be fastened to the base element so as to be pivotable about a pivot axis.

According to an advantageous embodiment of the invention, provision is made that the needle-guiding device has a clamping bracket which is fastened to the base element and is pivotable relative to the base element about the same pivot axis as the retaining bracket. The clamping bracket can form part of the aforementioned clamping mechanism. For example, the clamping bracket can be coupled to the retaining bracket such that, during a movement of the retaining bracket relative to the base element, the clamping bracket also performs the same movement, for example the aforementioned pivoting movement. The clamping bracket can be arranged, for example, between the retaining bracket and the base element. The clamping bracket can, for example, be arch-shaped, e.g. with an arch shape that is concentric to the arch shape of the retaining bracket.

The retaining bracket and the clamping bracket can, for example, be at least substantially parallel to each other, i.e. form two arches that run substantially parallel to each other. A certain distance or free space can be present between the retaining bracket and the clamping bracket. By virtue of the arch shape of at least the clamping bracket, the needle-guiding device has a free space in the region between the clamping bracket and the base element, said free space being, for example, substantially circular or, with respect to the pivoting possibility of the clamping bracket, hemispherical. The clamping bracket can be coupled to the retaining bracket in terms of pivotability, such that the clamping bracket is always pivoted by the same angle as the retaining bracket.

When fixing the needle-guiding element in the two degrees of angular freedom by means of the operating element, it is possible for the retaining bracket and the clamping bracket to be braced against each other, for which purpose the free space between the retaining bracket and the clamping bracket can be used. Retaining bracket and/or clamping bracket can be slightly deformed in the process. The clamping on the base element can take place, for example, at arch-shaped regions of the base element on which the retaining bracket and/or the clamping bracket are pivotably mounted. In this way, a kind of “remote clamping” can take place through actuation of the one operating element, i.e. a clamping effect is also triggered at a point remote from the operating element.

As has been mentioned, the needle-guiding device can be designed in such a way that, with each setting of the angular orientation of the needle-guiding element in the two degrees of angular freedom, a needle-shaped device guided through the needle-guiding element always comes into contact with the patient's body at the same position, i.e., in the case of a biopsy, the biopsy needle always hits the same puncture point. On a side facing the patient, the base element can have a fastening surface, with which the base element is to be fastened to the patient. For this purpose, the needle-guiding device can be designed in such a way that both the pivot axis of the retaining bracket (X axis) with respect to the base element and the pivot axis of the needle-guiding element (Y axis), defined by the arch shape of the retaining bracket, with respect to the base element run exactly in the plane of the fastening surface.

According to an advantageous embodiment of the invention, provision is made that a first angle scale is arranged on the retaining bracket and/or the clamping bracket, and/or a second angle scale is arranged in the region of a fastening arrangement of the retaining bracket on the base element. This has the advantage that existing, relatively large surfaces of these components (retaining bracket, clamping bracket and base element) can be used for labeling with a respective angle scale. In this way, the angle scale is easy to read. The first angle indicator, assigned to the first angle scale, can be arranged for example on the needle-guiding element or on a part connected to the needle-guiding element, for example the aforementioned retaining carriage. The second angle indicator, assigned to the second angle scale, can be arranged for example on the retaining bracket and/or the clamping bracket.

It is also advantageously possible to change around the abovementioned assignment of the first angle scale to the retaining bracket and/or the clamping bracket with the associated angle indicator. In other words, the first angle indicator can also be arranged on the retaining bracket and/or the clamping bracket. In this case, for example, the first angle scale, assigned to the first angle indicator, can be arranged on the needle-guiding element or on a component coupled to the needle-guiding element. This also applies correspondingly to the second angle scale. Thus, as an alternative, the second angle indicator can be arranged on the base element in the region of a fastening arrangement of the retaining bracket. In this case, for example, the second angle scale, assigned to the second angle indicator, can be arranged on the retaining bracket and/or the clamping bracket.

According to an advantageous embodiment of the invention, provision is made that the needle-guiding device has a needle-guiding element receptacle, designed to receive differently designed needle-guiding elements of the needle-guiding device. This has the advantage that the needle-guiding device can be used in a very versatile manner for a wide variety of examinations and/or treatments on patients. For example, by exchange of a needle-guiding element, the needle-guiding device can be adapted to needle-shaped devices having different diameters.

According to an advantageous embodiment of the invention, provision is made that the needle-guiding element receptacle extends from the retaining bracket to the clamping bracket. In this way, the retaining bracket can be coupled to the clamping bracket via the needle-guiding element receptacle. Accordingly, the entire needle-guiding device can be realized with very few components, since the needle-guiding element receptacle can fulfill several functions, namely the reception of differently designed needle-guiding elements and the coupling between the retaining bracket and the clamping bracket. Moreover, a further part of the clamping mechanism, for example a thread, can be arranged on the needle-guiding element receptacle.

According to an advantageous embodiment of the invention, provision is made that the operating element is designed as an annular operating element with an inner cavity, at least part of the needle-guiding element extending through the inner cavity. The operating element can be designed, for example, in the manner of a nut with an internal thread. The internal thread can be a thread that matches the thread of the needle-guiding element receptacle, such that the operating element can be screwed to the needle-guiding element receptacle in the manner of a nut. Through this threaded connection between the operating element and the needle-guiding element receptacle, the clamping mechanism can be activated, for example by tightening the threaded connection to obtain the clamping in order to fix the needle-guiding element relative to the base element in the two degrees of angular freedom. Moreover, a particularly compact construction of the needle-guiding device can be realized in this way.

According to an advantageous embodiment of the invention, provision is made that the base element has a bearing surface for bearing on the patient's body. The bearing surface can, for example, have a narrow, elongate rectangular shape, optionally with rounded corners. In this way, the needle-guiding device requires only little space for the bearing surface on the patient's body, such that it can be used in a very versatile manner, for example even on small patients. The bearing surface can be designed as a flat surface.

The object mentioned at the outset is moreover achieved by a set comprising a plurality of elements for carrying out an examination and/or treatment of a patient, the set having at least the following elements:
a) a needle-guiding device for guiding and positioning a needle-shaped device on the patient, the needle-guiding device having a base element and a needle-guiding element on which the needle-shaped device is guidable along its longitudinal extent, the angular orientation of the needle-guiding element relative to the base element being adjustable in at least one degree of angular freedom or several mutually independent degrees of angular freedom, e.g. in two or three mutually independent degrees of angular freedom,
b) a computer with a computer program, or at least one such computer program for a computer, the computer program being set up to use
   characteristic data of the needle-guiding device,
   examination data of the patient and
   at least one predetermined examination and/or treatment step to be carried out with the needle-guiding device,
   in order to calculate
   b1) positioning specifications for positioning of the needle-guiding device (1) on the patient and/or
   b2) one or more angular orientation specifications for setting the angular orientation of the needle-guiding element relative to the base element in the at least one degree of angular freedom or the several mutually independent degrees of angular freedom
   and to output these specifications to the user when the computer program is run on the computer.

In short, the set thus has at least two elements, namely the needle-guiding device and the computer with a computer program, or the computer program itself. The computer program can be stored on a data carrier. The set can also have one or more additional elements, for example the orientation aid explained below.

Such a set supports the physician in carrying out the various steps of the examination and/or treatment, for example in the case of a biopsy. In particular, the process sequence can be supported by the computer program, by means of the computer program or the computer outputting corresponding instruction steps for the individual method steps that are to be carried out.

The computer is set up to execute the computer program, e.g. in the sense of software. The computer can be designed as a commercially available computer, e.g. as a PC, laptop, notebook, tablet or smartphone, or as a microprocessor, microcontroller or FPGA, or as a combination of such elements.

Advantageously, the computer or the computer program supports the user in particular when positioning the needle-guiding device on the patient and/or when setting the angular orientation of the needle-guiding element relative to the base element in the at least one degree of angular freedom or the several independent degrees of angular freedom. In other words, the computer program can make it much easier for the user to correctly position and adjust the needle-guiding device.

The computer program requires, as input, at least characteristic data of the needle-guiding device, for example specifications concerning the geometry of an angle adjustment mechanism and the arrangement of reference points of the base element. The characteristic data of the needle-guiding device can be stored in advance in the computer, for example, and, when different needle-guiding devices are used, the characteristic data of these different needle-guiding devices can be stored in the computer and a selection can be displayed for the user, such that the user, by input on the computer, can select the particular needle-guiding device used.

The computer program also requires, as input, examination data of the patient and at least one predetermined examination and/or treatment step to be carried out with the needle-guiding device. The examination data of the patient can be data from an imaging examination of the patient, for example from an X-ray, MRT and/or ultrasound examination. The X-ray examination can be, for example, a CT examination. For example, the image data obtained therefrom or the DICOM files can be fed directly to the computer for processing by means of the computer program. The specifications relating to the examination or treatment step to be carried out can be, for example, the user-defined coordinates of a puncture point of the needle-shaped device on the patient and also of a target point lying in the body.

The input data mentioned can be fed to the computer via a wireless or wired interface and/or via manual entries.

The computer program then generates, as output, the aforementioned positioning specifications and/or angular orientation specifications. For example, the two-dimensional coordinates in a patient-related coordinate system for at least two reference points, which can be located for example on the base element of the needle-guiding device, are output as positioning specifications. As regards the angular orientation specifications, two angle specifications can be output, for example in the unit of degrees, based on a 360 degree system. The user can then use these positioning specifications and/or angle orientation specifications to place the needle-guiding device at the desired location and in the desired position on the body and can adjust the angular orientation of the needle-guiding element in the two degrees of angular freedom before or after this placement. The needle-guiding device contained in the set can be, for example, a needle-guiding device of the type described at the outset, that is to say a needle-guiding device according to the invention, or another type of needle-guiding device, for example a known needle-guiding device. For example, the needle-guiding device according to WO 2019/101 862 A1 can also be used.

According to an advantageous embodiment of the invention, provision is made that the set has, as a further element, an orientation aid on which markings are placed which define a two-dimensional coordinate grid, the orientation aid having a fastening surface which is configured to fasten the orientation aid to the patient. Such an orientation aid makes the assignment of a patient-related coordinate system to the coordinate system used by the computer program safer and simpler. In particular, such an assignment is simplified for the user, since only the orientation aid has to be applied to the patient, and, by automatic detection of the orientation aid on the patient, for example by means of a camera and image evaluation, corresponding characteristic data of the patient-related coordinate system can be made available to the computer and the computer program.

The orientation aid can consist, for example, of several strips which are arranged at right angles to one another and are joined together to form a grid. Markings or scales can be applied to the individual strips, can be read by the user and facilitate optical recognition of a desired coordinate position on the patient. The orientation aid can also have a substantially closed surface, similar to a sheet of paper or a large plaster on which the two-dimensional coordinate grid is applied, for example by printing.

The coordinate grid can, for example, be printed on the orientation aid or applied in some other way. The coordinate grid can be designed in the sense of a Cartesian coordinate system and can, for example, have a horizontal coordinate (X coordinate) and a vertical coordinate (Y coordinate). The coordinate axes can be labeled with numbers and/or letters, for example.

According to an advantageous embodiment of the invention, provision is made that the orientation aid is designed as a flat flexible element. This has the advantage that the orientation aid can also be easily applied to the patient in the region of curves of the body and protuberances.

According to an advantageous embodiment of the invention, provision is made that the examination data of the patient include positional information relating to the orientation aid fastened to the patient, the computer program being set up to calculate the output of the positioning specifications and/or of the angular orientation specifications additionally as a function of the positional information relating to the orientation aid on the patient. Accordingly, the “examination data” input additionally contains the positional information relating to the orientation aid fastened to the patient's body. This positional information can be automatically evaluated by the computer program, such that the positioning specifications and/or the angular orientation specifications can be output directly in relation to the patient-related coordinate system defined by the orientation aid.

According to an advantageous embodiment of the invention, provision is made that the orientation aid has a plurality of marker elements which are automatically detectable using X-ray, MRT and/or ultrasound images. These marker elements can be arranged spatially distributed on the orientation aid. For example, the marker elements can be arranged in a matrix-like manner over the orientation aid. It is also possible for only individual points of the orientation aid to be equipped with marker elements, for example two or three points. This too allows automatic evaluation of the positioning of the orientation aid on the patient on the basis of the X-ray, MRT and/or ultrasound images.

If there is a larger number of marker elements distributed in a matrix-like manner, the marker elements can also be used to automatically detect the surface contour of the patient's body in the region of the orientation aid using the X-ray, MRT and/or ultrasound images. As an alternative or in addition, such a detection of the surface contour can also be carried out directly by image processing of the X-ray, MRT and/or ultrasound images, without such a matrix of marker elements being present. An automatic detection of the surface contour is also possible by means of a separate detection device arranged in the region of the patient, for example by means of a camera, a stereoscopic camera, a laser scanner or comparable detection instruments, and these can also be used in combination with one another.

The object mentioned at the outset is moreover achieved by a set comprising a plurality of elements for carrying out an examination and/or treatment of a patient, the set having at least the following elements:
a) an orientation aid on which markings are placed which define a two-dimensional coordinate grid, the orientation aid having a fastening surface which is configured to fasten the orientation aid to the patient,
b) a computer with a computer program, or at least one such computer program for a computer, the computer program being set up to use
   characteristic data of the orientation aid,
   examination data of the patient and
   at least one position on the patient, selected by the user from the examination data of the patient,
   in order to calculate coordinate specifications in the coordinate grid of the orientation aid and output said specifications to the user when the computer program is run on the computer.

In short, the set thus has at least two elements, namely the orientation aid and the computer with a computer program, or the computer program itself. The computer program can be stored on a data carrier. The set can also have one or more additional elements, for example the needle-guiding device. The orientation aid can be developed in accordance with one or more of the variants explained above.

Such a set supports the physician in carrying out the various steps of the examination and/or treatment, for example in the case of a biopsy. Advantageously, the computer or the computer program supports the user in particular in finding a desired real position on the patient.

The computer program requires, as input, at least characteristic data of the orientation aid, for example specifications concerning the size and distribution of the coordinate grid and optionally the position of marker elements. The characteristic data of the orientation aid can be stored in advance in the computer, for example, and, when different orientation aids are used, the characteristic data of these different orientation aids can be stored in the computer and a selection can be displayed for the user, such that the user, by input on the computer, can select the particular orientation aid used.

The computer program moreover requires, as input, examination data of the patient, which can be obtained as already explained above, and at least one position on the patient selected by the user from the examination data of the patient. The user, for example the physician, can for example select a position on the patient on the computer in the MRT images shown on a display, e.g. a desired puncture site during the biopsy.

The computer program then generates, as output, the aforementioned coordinate specifications in the coordinate grid of the orientation aid, e.g. an X coordinate and a Y coordinate, and outputs these, for example, on the display.

The object mentioned at the outset is moreover achieved by a computer program for a computer, the computer program being set up to use
characteristic data of the needle-guiding device,
examination data of the patient and
at least one predetermined examination and/or treatment step to be carried out with the needle-guiding device
in order to calculate
b1) positioning specifications for positioning of the needle-guiding device on the patient
and/or
b2) one or more angular orientation specifications for setting the angular orientation of the needle-guiding element relative to the base element in the at least one degree of angular freedom or the several mutually independent degrees of angular freedom
   and to output these specifications to the user when the computer program is run on the computer.

The advantages explained above can also be realized in this way.

The object mentioned at the outset is moreover achieved by a computer program for a computer, the computer program being set up to use
characteristic data of the orientation aid,
examination data of the patient and
at least one position on the patient, selected by the user from the examination data of the patient,
   in order to calculate coordinate specifications in the coordinate grid of the orientation aid and output said specifications to the user when the computer program is run on the computer.

The advantages explained above can also be realized in this way. The invention also relates to a combined computer program which contains the functions of the two aforementioned computer programs.

The invention is explained in more detail below on the basis of exemplary embodiments and with reference to the drawings, in which:

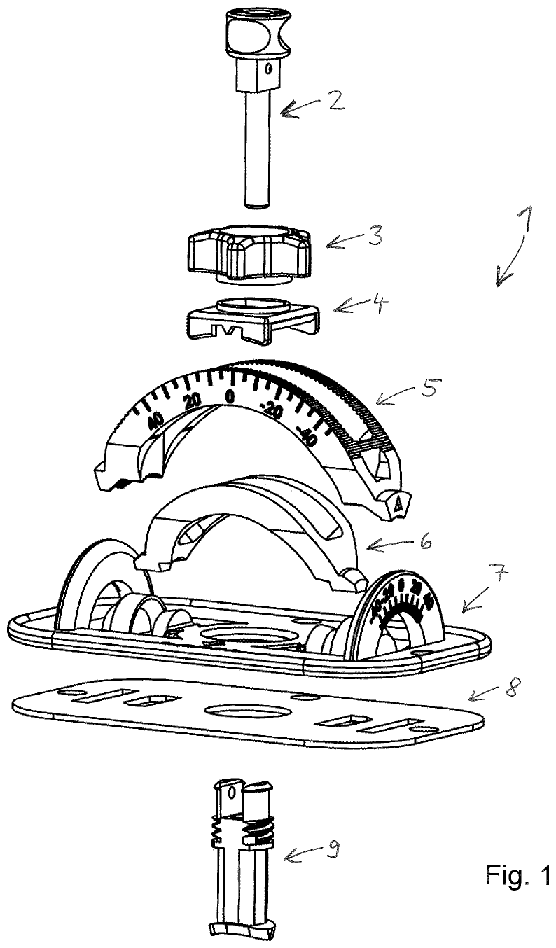

FIG. 1 shows a needle-guiding device in an exploded view,

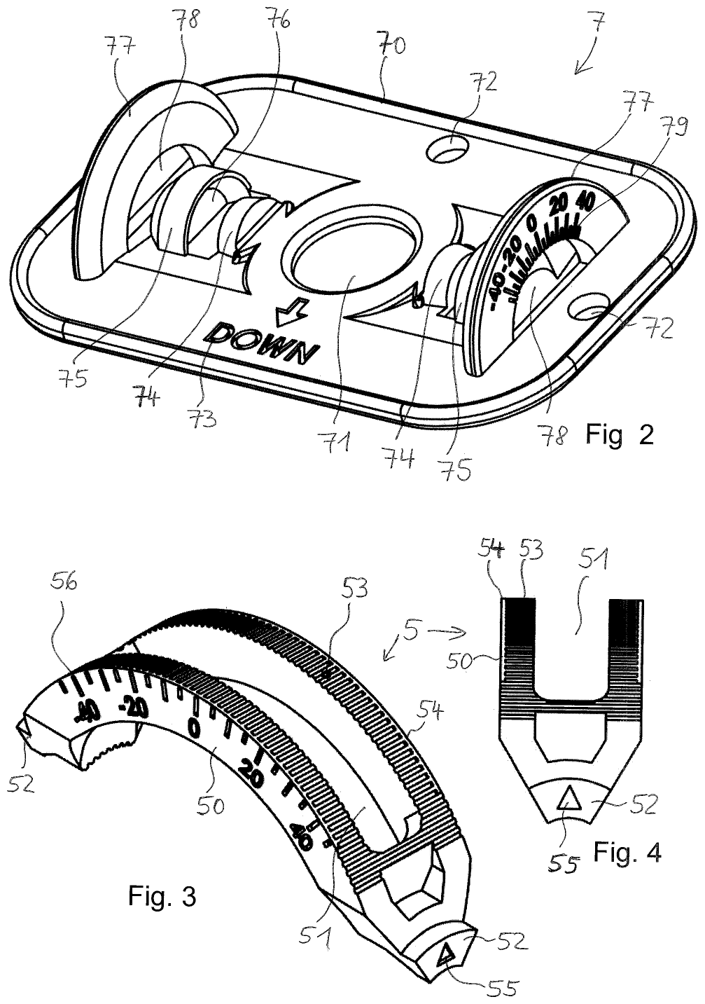

FIG. 2 shows a base element,

FIGS. 3, 4 show a retaining bracket,

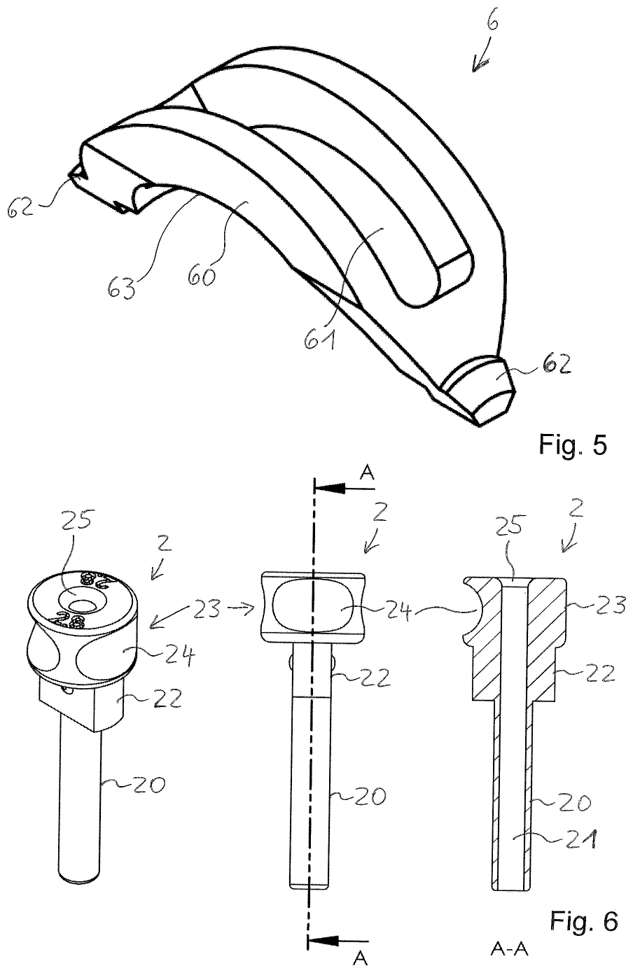

FIG. 5 shows a clamping bracket,

FIG. 6 shows a needle-guiding element,

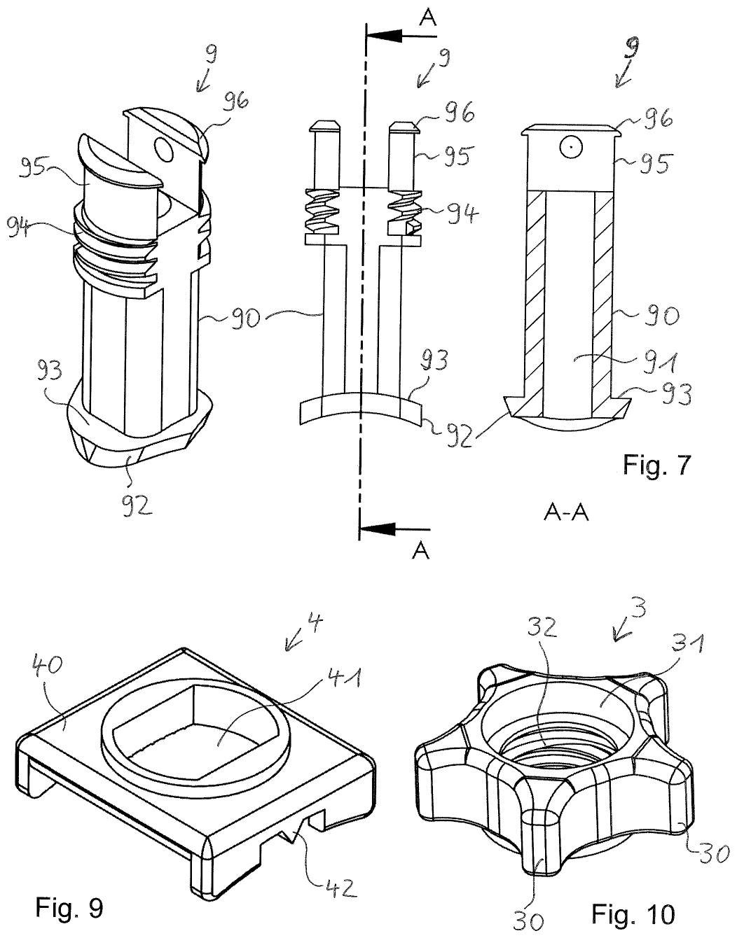

FIG. 7 shows a needle-guiding element receptacle,

FIG. 9 shows a retaining carriage,

FIG. 10 shows an operating element,

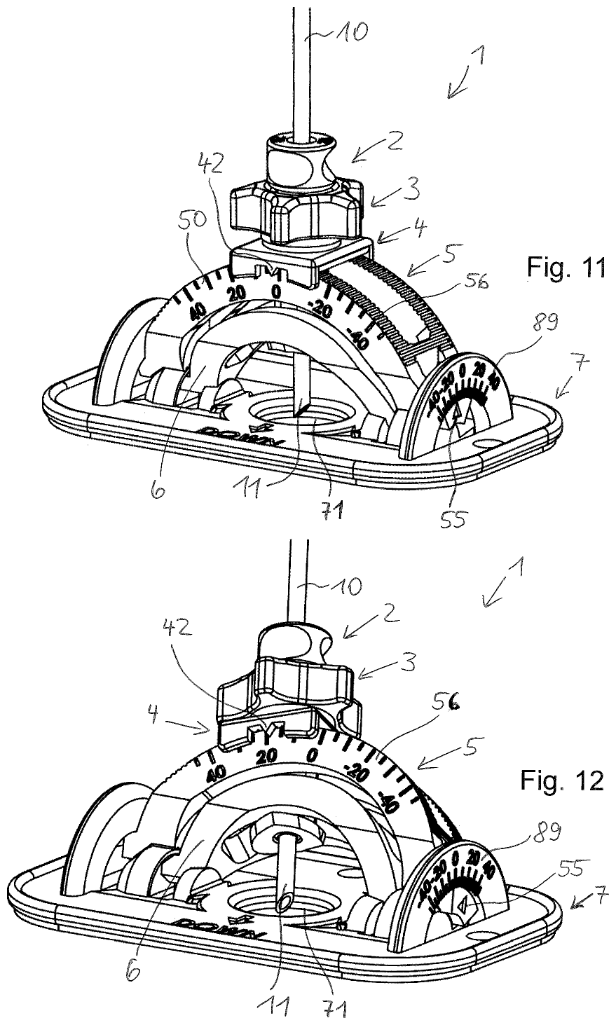

FIGS. 11, 12 show the needle-guiding device in different angular settings,

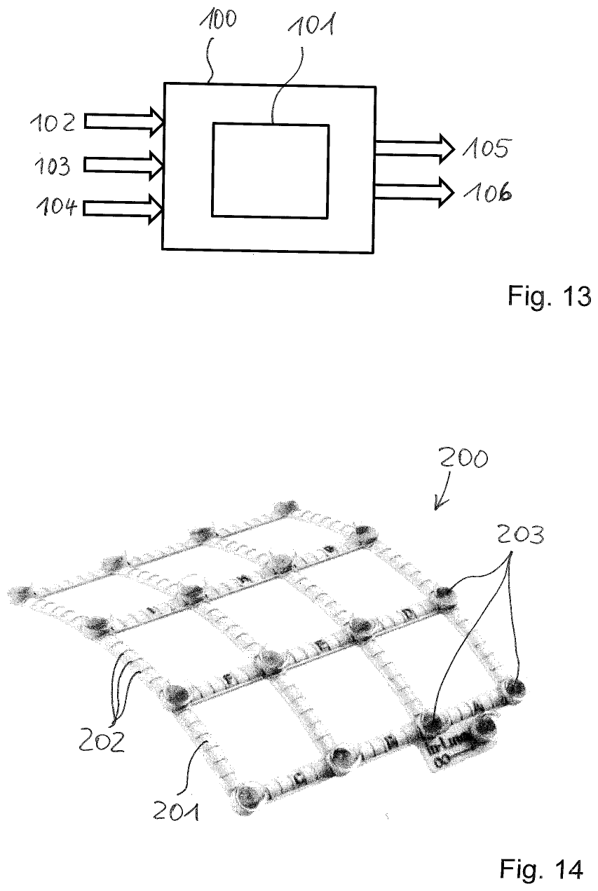

FIG. 13 shows a computer with a computer program and

FIG. 14 shows an orientation aid and

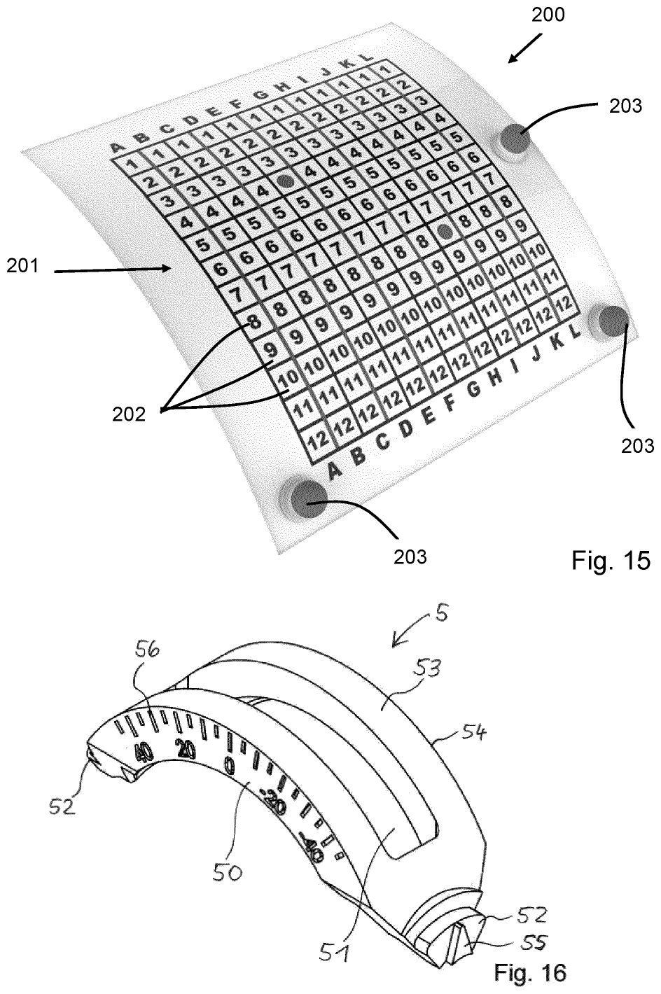

FIG. 15 shows a further embodiment of an orientation aid and

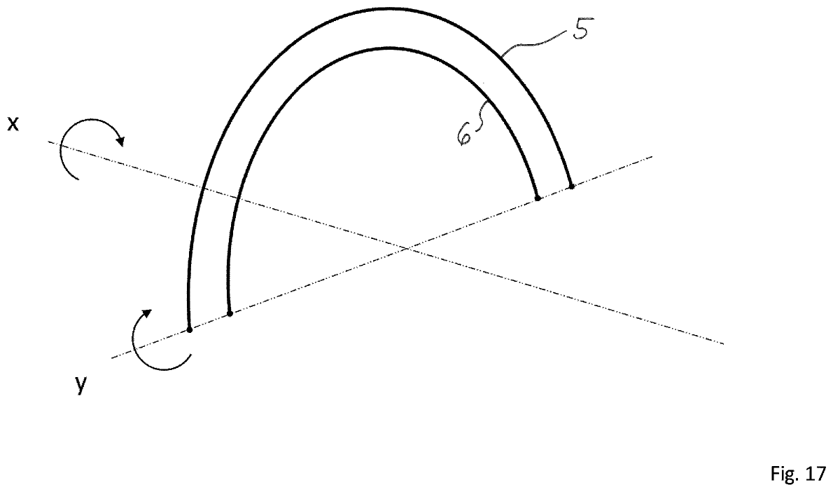

FIG. 16 shows a further embodiment of a retaining bracket and

FIG. 17 shows a highly schematic representation of the retaining bracket and of the clamping bracket and

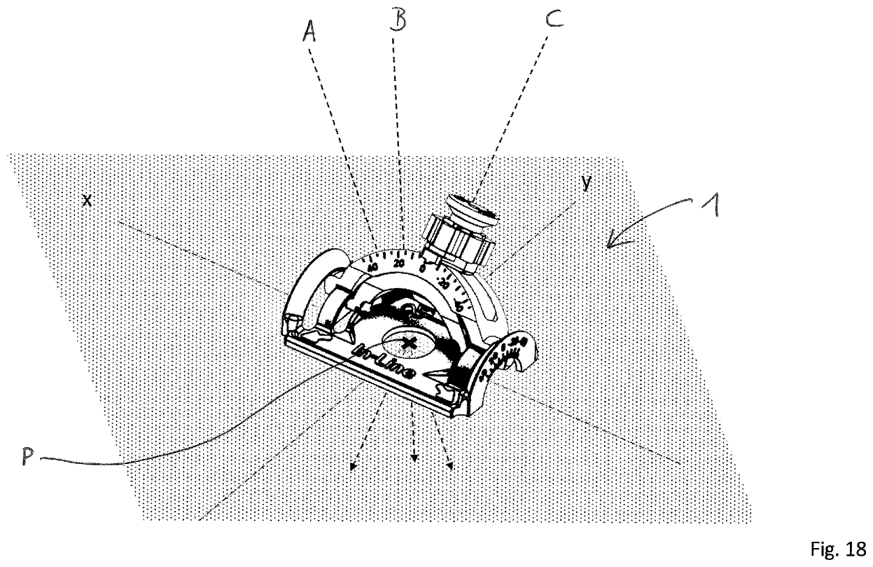

FIG. 18 shows the needle-guiding device in various angular settings and

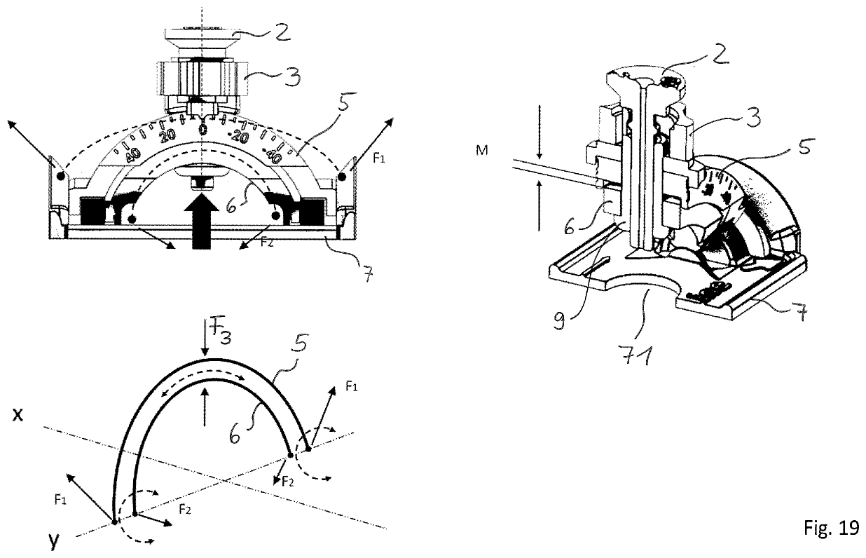

FIG. 19 shows an explanation of the clamping mechanism.

The reference signs used in the figures denote the following:
1 needle-guiding device
2 needle-guiding element
3 operating element
4 retaining carriage
5 retaining bracket
6 clamping bracket
7 base element
8 contact element
9 needle-guiding element receptacle
10 needle-shaped device
11 tip
20 needle-guiding body
21 through-channel
22 connection region
23 handling region
24 grip recesses
25 insertion opening
30 grip elements
31 cavity
32 internal thread
40 main body
41 opening
42 second angle indicator
50 main body
51 opening
52 bearing element
53 outer surface
54 edge region
55 first angle indicator
56 second angle scale
60 main body
61 opening
62 bearing element
70 main body
71 needle feed-through opening
72 position marking
73 marking
74 clamping arch
75 bearing arch
76 recess
77 retaining arch
78 recess
79 first angle scale
90 longitudinal portion
91 through-opening
92 region
93 bearing face
94 threaded region
95 retaining portion
96 fixing element
100 computer
101 computer program
102 characteristic data
103 examination data
104 data of an examination and/or treatment step
105 positioning specifications
106 angular orientation specifications
200 orientation aid
201 grid structure
202 marking
203 marker element

The needle-guiding device 1 shown in FIG. 1 has a needle-guiding element 2, an operating element 3, a retaining carriage 4, a retaining bracket 5, a clamping bracket 6, a base element 7, a contact element 8, and a needle-guiding element receptacle 9. The contact element 8 is optional. It is used, for example, to fasten the base element 7 to the body of a patient. For this purpose, the contact element 8 can be provided with an adhesive, for example. Alternatively, the underside of the base element 7 can be brought into direct contact with the body of the patient. In this case, an adhesive can be arranged there. Overall, the needle-guiding device 1 can thus be composed of only seven components 2, 3, 4, 5, 6, 7 and 9, which can be formed entirely or predominantly as plastic components.

These components are explained in sequence below.

FIG. 2 firstly shows the base element 7 in a perspective view. The base element 7 has a main body 70 which can be designed in the manner of a flat, planar plate. On the underside of the main body 70, the base element 7 can have a bearing face for bearing on the body of the patient. The aforementioned contact element 8 can also be applied there.

The main body 70 has a needle feed-through opening 71 which is used to feed the needle-shaped device through to the body of the patient. In a biopsy for example, the puncture site of the biopsy needle can be arranged in the region of the needle feed-through opening 71. Moreover, the main body 70 has a plurality of position markings 72, for example in the form of through-holes. By means of these position markings 72, the needle-guiding device 1 can be precisely positioned on the patient in accordance with a patient-related coordinate system.

In addition, the main body 70 can have further markings 73, for example information on a reference position, for example the underside of the needle-guiding device 1.

On both sides of the needle feed-through opening 71, retaining arches 77 protrude from the main body 70 and serve to hold and pivot the retaining bracket 5 on the base element 7. The retaining arches 77 have respective circular arc-shaped or semicircular recesses 78 on which the retaining bracket 5 can be pivotably mounted. Adjacent to a respective retaining arch 77, a bearing arch 75 protrudes from the base body 70. The outwardly facing curved surface of the bearing arch 75 also serves to form a pivot bearing for the retaining bracket 5. The bearing arches 75 also have inner recesses 76, which serve as part of a pivot bearing of the clamping bracket 6. Adjacent to the bearing arches 75, for example on each side of the needle feed-through opening 71, a clamping arch 74 is arranged on the main body 70. The curved outer surface of the clamping arch 74 serves as a further part of the pivot bearing for the clamping bracket 6. As can be seen, starting from the center of the main body 70 or from the needle feed-through opening 71, there is firstly a clamping arch 74 on each side, then a bearing arch 75 and, finally, a retaining arch 77.

It can also be seen that a first angle scale 79 can be arranged on the surface of the retaining arch 77 facing away from the needle feed-through opening 71.

FIG. 3 shows the retaining bracket 5 in a perspective view, while FIG. 4 shows it in a side view. The retaining bracket 5 has a main body 50 which is arc-shaped and has a slit-like opening 51 which runs in the longitudinal direction and through which the needle-guiding element 2 or the needle-guiding element receptacle 9 can be guided. At each of the free ends remote from one another, the retaining bracket 5 has a bearing element 52 which, in terms of its curved outer contours, is adapted to the inner contour of the opening 78, and to the outer contour of the bearing arch 75 facing toward the retaining arch 77. In this way, the retaining bracket 5, placed on the base element 7 with its bearing element 52 between the retaining arch 77 and the bearing arch 75, can be pivoted steplessly about a pivot axis formed in this way. Arranged on the bearing element 52 is a first angle indicator 55, which is assigned to the first angle scale 79. By the display of the first angle indicator 55 on the first angle scale 79, the angular orientation of the needle-guiding element 2 relative to the base element 7 can be read in a first degree of angular freedom.

It can also be seen from FIGS. 3 and 4 that the retaining bracket 5 can have a structuring, for example a corrugation, on the convexly curved outer surfaces 53. The outer surfaces 53 serve as a supporting and sliding surface for the retaining carriage 4. However, in order to ensure that the retaining carriage 4 can be adjusted as steplessly as possible on the outer surfaces 53, the surface structuring or the corrugation can be sunk slightly in relation to an edge region 54 of the outer surfaces 53, such that the surface structuring or corrugation only becomes effective when the clamping mechanism is fixed.

It can also be seen that a second angle scale 56 can be arranged on the retaining bracket 5.

FIG. 5 shows the clamping bracket 6 in a perspective view. The clamping bracket 6 is shaped similarly to the retaining bracket 5. The clamping bracket 6 has a main body 60, which extends in an arc shape and has a slit-like opening 61 extending in the longitudinal direction. The needle-guiding element 2 or the needle-guiding element receptacle 9 can be guided through the slit-like opening 61. On the concave inner surface 63 of the arc-shaped main body 60, a retaining surface is formed which serves as a counter-bearing for the needle-guiding element receptacle 9, as will be explained below.

Similar to the retaining bracket 5, the clamping bracket 6, at each of the free ends thereof remote from one another, has a bearing element 62 with which the clamping bracket 6 can be placed in the opening 76 of the base element 7. The bearing element 62 is then located between an inside of the bearing arch 75 and an outer surface of the clamping arch 74. In this way, a pivot bearing for pivoting the clamping bracket 6 is formed. The pivot axes of the clamping bracket 6 and of the retaining bracket 5 are identical.

FIG. 6 shows the needle-guiding element 2 on the left in a perspective view, in the middle in a side view, and on the right in a sectional view in the section plane AA. The needle-guiding element 2 has a needle-guiding body 20, which can be designed, for example, as a hollow cylindrical body with an inner through-channel 21. The inner through-channel 21 extends completely through the needle-guiding body 20 in the longitudinal direction. The needle-shaped device, for example a biopsy needle, can be guided through the through-channel 21. Above the needle-guiding body 20, the needle-guiding element 2 has a connection region 22 in which the needle-guiding element 2 can be coupled to the needle-guiding element receptacle 9. Above the connection region 22, the needle-guiding element 2 has a manual handling region 23 which, for example, can have one or more grip recesses 24, which make it easier to grip and manually insert or remove the needle-guiding element 2 from the needle-guiding device 1. The inner through-channel 21 also extends in the longitudinal direction through the connection region 22 and the manual handling region 23. At the upper end of the needle-guiding element 2, the inner through-channel 21 opens into an insertion opening 25 at which the needle-shaped device can be inserted into the needle-guiding element 2.

FIG. 7 shows the needle-guiding element receptacle 9 on the left in a perspective view, in the middle in a side view, and on the right in a sectional view in the section plane A-A. The needle-guiding element receptacle serves to accommodate differently designed needle-guiding elements 2. As a common interface to the differently designed needle-guiding elements 2, the needle-guiding element receptacle 9 has a longitudinally extending through-opening 91 which, its terms of is dimensions, is adapted to the external dimensions of the needle-guiding body 20 of the needle-guiding element 2. The needle-guiding element 2 is thus inserted with the needle-guiding body 20 into the through-opening 91. When the needle-guiding element 2 is completely inserted into the needle-guiding element receptacle 9, the connection region 22 is located within a retaining portion 95 of the needle-guiding element receptacle 9 at which the needle-guiding element 2 is fixed on the needle-guiding element receptacle 9. The retaining portion 95 can be designed in the form of two longitudinally protruding retaining tabs which are in the shape of an at least partial circular arc on the circumferential side. This circular arc-shaped circumferential contour at the same time provides a rotational position for the operating element 3.

The retaining portion 95 can terminate at the free end with a circumferentially protruding fixing element 96, for example similar to a mushroom head shape. In this way, the operating element 3 can be captively coupled to the needle-guiding element receptacle 9. The operating element 3 is simply latched into place by being pushed onto the retaining portion 95 and fixed thereon by the protruding fixing elements 96. Below the retaining portion 95, the needle-guiding element receptacle 9 has a threaded region 94 which is designed as a thread that matches an internal thread of the operating element 3. Located below the threaded region 94 is a longitudinal portion 90 of the needle-guiding element receptacle 9 which, in the fully assembled state of the needle-guiding device 1, produces a connection between the retaining bracket 5 and the clamping bracket 6. Below the longitudinal portion 90, the needle-guiding element receptacle 9 transitions into a region 92 with an enlarged cross-sectional area, which region 92 forms an arc-shaped bearing surface 93 at least in one viewing direction (FIG. 7: middle). In the fully assembled state of the needle-guiding device 1, the arc-shaped bearing surface 93 comes into contact with the concave inner surface 63 of the clamping bracket 6. In this way, part of the movable pivot bearing of the needle-guiding element 2 is formed on the retaining bracket 5 and the clamping bracket 6.

FIG. 9 shows the retaining carriage 4 in a perspective view. The retaining carriage 4 has a main body 40 which has an opening 41. A part of the needle-guiding element receptacle 9 can be inserted through the opening 41, in particular the upper part of the longitudinal portion 90, which transitions into the threaded region 94. On the underside, the retaining carriage 4 has bearing surfaces via which the retaining carriage 4 is guided on the convexly curved outer surfaces 53 of the retaining bracket 5 and slides thereon or, upon activation of the clamping mechanism, is clamped thereon. The second angle indicator 42, which serves to indicate the angle on the second angle scale 56 of the retaining bracket 5, is also arranged on the retaining carriage 4.

FIG. 10 shows the operating element 3, which can be designed in the form of a nut with an internal thread 32. The internal thread 32 can be screwed onto the threaded region 94 of the needle-guiding element receptacle 9. The operating element 3 has an inner cavity 31 through which at least part of the needle-guiding element 2 can be inserted. On the outer circumference, the operating element 3 can have grip elements 30 which are suitable for improving the transmission of an operating force, for example the projections shown, or some other surface structuring and/or corrugation on the outer circumference.

FIG. 11 shows the assembled needle-guiding device 1 with the elements already mentioned, and a needle-shaped device 10 is shown at least in part. The needle-shaped device 10 terminates with a tip 11 in the region of the through-opening 71 of the base element 7. FIG. 11 shows the needle-guiding device 1 in a setting in which the angular orientation of the needle-guiding element 2 is neutral with respect to both adjustable degrees of angular freedom, that is to say the respective angle indicators 42, 55 point to the zero value of the respective angle scale 56, 79. In FIG. 12, the angular orientation has been adjusted in such a way that, for example, the second angle indicator 42 points to an angle of ca. −20 degrees on the second angle scale 56. The first angle indicator 55 points to an angle of ca. +30 degrees on the first angle scale 79.

To adjust the needle-guiding device 1 from one angular orientation to another angular orientation, the operating element 3 simply has to be rotated slightly about its longitudinal axis, for example in the leftward direction of rotation, as a result of which the threaded connection between the internal thread 32 and the threaded region 94 is slightly adjusted, and the clamping effected by the clamping bracket 6, which can be tensioned via the threaded connection and the needle-guiding element receptacle 9, is thereby canceled. When the desired angular orientation is set, the operating element 3 simply has to be rotated in the opposite direction of rotation, as a result of which a tensioning force is exerted on the clamping bracket 6 via the threaded connection 32, 94 and the needle-guiding element receptacle 9, and the clamping bracket 6 is braced with respect to the retaining bracket 5. As a result, all the degrees of freedom of pivoting and of movement of the angular adjustment of the needle-guiding device 1 are fixed.

FIG. 13 shows, as part of a set having a plurality of elements for carrying out an examination and/or treatment on a body of a patient, a computer 100 which has a computer program 101. The set can include any desired needle-guiding device, for example a needle-guiding device of the type described above, and/or an orientation aid of the type described above.

In the computer 100 or in the computer program 101, input data are entered as characteristic data 102 of the needle-guiding device contained in the set, examination data 103 of a patient, and data 104 of a predetermined examination and/or treatment step to be carried out by means of the needle-guiding device. From this, the computer program calculates positioning specifications 105 for positioning the needle-guiding device on the patient, and also angular orientation specifications 106 for setting the angular orientation of the needle-guiding element relative to the base element in the two mutually independent degrees of angular freedom. The output positioning specifications and angular orientation specifications can be shown, for example, on a display of the computer 100.

The aforementioned set can have an orientation aid 200, for example the orientation aid 200 shown in FIG. 14. The orientation aid 200 has a grid-shaped structure 201 which can be formed from flat, flexible sub-elements. Markings 202 for defining a two-dimensional coordinate grid can be arranged on these sub-elements. In addition, marker elements 203 can be arranged on the orientation aid 200 at several points, for example in the form of a matrix. The marker elements 203 are formed from a material which can be detected in X-ray, MRT and/or ultrasound examinations and can accordingly also be automatically evaluated by image processing.

FIG. 15 shows a further embodiment of an orientation aid 200. While the embodiment according to FIG. 14 has a grid-shaped structure of cross-connected elongate elements, FIG. 15 shows an embodiment in which the grid-shaped structure 201 is applied to an at least substantially closed main body, for example a sheet of paper or a film. The grid-shaped structure 201, together with the markings 202 arranged thereon, can be printed on, for example. The aforementioned marker elements 203 can also be present in this orientation aid 200.

If the aforementioned itself has only the orientation aid 200 and the computer 100 or the computer program 101, the computer program 101 can be designed in such a way that the only input data needed are characteristic data of the orientation aid 200, examination data of the patient, and at least one position on the patient as selected by the user from the examination data of the patient. From this, the computer program 101 calculates coordinate specifications in the coordinate grid of the orientation aid and outputs these to the user, for example the specification E 4 for a position in the fourth line and the fifth column.

FIG. 16 shows an alternative embodiment of the retaining bracket 5 in which, in contrast to the embodiment described thus far, there is no special structuring on the convexly curved outer surfaces 53, and instead these outer surfaces 53 are designed with the relatively smooth surface normal for the material used.

FIG. 17 shows, in a highly schematic manner, the retaining bracket 5 and the clamping bracket 6 and also the mutually orthogonal pivot axes X, Y, in which the needle-guiding device 1 can be adjusted in different angular orientations. It will be seen that the retaining bracket 5 and the clamping bracket 6 each have substantially the shape of an arch, these arches running substantially parallel to each other. The retaining bracket 5 is located above the clamping bracket 6. Both the retaining bracket 5 and the clamping bracket 6 are pivotable about the Y-axis. Since no further arch is present in this region for the pivotability about the X-axis, a free space is present below the clamping bracket 6.

FIG. 18 uses three different angular orientations A, B, C, in which the needle-guiding device 1 can be set, to show that with each setting the needle-shaped device is always guided by the needle-guiding element 2 through the same point P, i.e. the needle-shaped device always has the same puncture point on the patient, irrespective of the angle setting.

FIG. 19 uses partial views of the needle-guiding device 1 (top) and a schematic view similar to FIG. 17 (bottom) to show the mode of operation of the clamping mechanism. The clamping is brought about via the operating element 3, i.e. by tightening the nut with the internal thread 32 in relation to the threaded region 94 of the needle-guiding element receptacle 9. In this way, the retaining bracket 5 is braced relative to the clamping bracket 6, i.e. the tightening causes the clamping bracket 6 to be drawn slightly to the retaining bracket 5. The distance M shown is reduced here. The arch shape of the retaining bracket 5 is thereby slightly flattened, while the clamping bracket 6 is slightly more bent. As a result, the needle-guiding element 2 is first of all fixed with respect to the pivotability about the X-axis. Moreover, the retaining bracket 5 and the clamping bracket 6 are fixed on the base element 7 by the forces F1, F2 occurring in this transition region.

1. A needle-guiding device for guiding and positioning a needle-shaped device on a patient, comprising:
a base element,
a needle-guiding element on which the needle-shaped device is guidable along its longitudinal extent, wherein an angular orientation of the needle-guiding element relative to the base element is adjustable in two mutually independent degrees of angular freedom,
an operating element by which the angular orientation of the needle-guiding element is fixable in the two degrees of angular freedom, and by which the angular orientation of the needle-guiding element is releasable from a fixed position in two degrees of angular freedom,
a plurality of angle scales comprising a respective angle scale for each of the two adjustable degrees of angular freedom, and
an angle indicator assigned to each respective angle scale such that a currently set angular orientation of the needle-guiding element in the two degrees of angular freedom can be read off by a user on a basis of a position of the respective angle indicator in relation to the assigned angle scale.

2. The needle-guiding device as claimed in claim 1, further comprising a clamping mechanism that is actuatable by the operating element, wherein the angular orientation of the needle-guiding element relative to the base element in the two degrees of angular freedom is fixable by clamping through operation of the clamping mechanism by the operating element.

3. The needle-guiding device as claimed in claim 1 wherein the angular orientation of the needle-guiding element relative to the base element is steplessly adjustable in the two degrees of angular freedom.

4. The needle-guiding device as claimed in claim 1 further comprising a retaining bracket to which the needle-guiding element is fastenable, wherein the retaining bracket in a first degree of angular freedom of the two degrees of angular freedom is movable with respect to the base element, and wherein the needle-guiding element in a second degree of angular freedom of the two degrees of angular freedom is movable with respect to the retaining bracket.

5. The needle-guiding device as claimed in claim 4, further comprising a clamping bracket which is fastenable to the base element and is pivotable relative to the base element about a same pivot axis as the retaining bracket.

6. The needle-guiding device as claimed in claim 5 wherein a first angle scale of the plurality of angle scales is arranged on the retaining bracket and/or the clamping bracket, and/or a second angle scale of the plurality of angle scales is arranged in a region of a fastening arrangement of the retaining bracket on the base element.

7. The needle-guiding device as claimed in claim 1 wherein the operating element is designed as an annular operating element with an inner cavity, wherein at least part of the needle-guiding element extends through the inner cavity.

8. A set comprising a plurality of elements for carrying out an examination and/or treatment of a patient, comprising:
a) a needle-guiding device for guiding and positioning a needle-shaped device on the patient, the needle-guiding device comprising a base element and a needle-guiding element on which the needle-shaped device is guidable along its longitudinal extent, wherein an angular orientation of the needle-guiding element relative to the base element is adjustable in at least one degree of angular freedom or is adjustable in several mutually independent degrees of angular freedom,
b) a computer program with instructions encoded on a non-transient medium which are executable on a computer, the computer program being set up to use
characteristic data of the needle-guiding device,
examination data of the patient, and
at least one predetermined examination and/or treatment step to be carried out with the needle-guiding device
in order to calculate
b1) positioning specifications for positioning of the needle-guiding device on the patient
and/or
b2) one or more angular orientation specifications for setting the angular orientation of the needle-guiding element relative to the base element in the at least one degree of angular freedom or in the several mutually independent degrees of angular freedom,
and to output the one or more angular orientation specifications to the user when the computer program is run on the computer.

9. The set as claimed in claim 8, wherein the needle-guiding device further comprises
an operating element by which the angular orientation of the needle-guiding element is fixable in the two degrees of angular freedom, and by which the angular orientation of the needle-guiding element is releasable from a fixed position in two degrees of angular freedom,
a plurality of angle scales comprising a respective angle scale for each of the two adjustable degrees of angular freedom, and
an angle indicator assigned to each respective angle scale such that a currently set angular orientation of the needle-guiding element in the two degrees of angular freedom can be read off by a user on a basis of a position of the respective angle indicator in relation to the assigned angle scale.

10. The set as claimed in claim 8 wherein the examination data of the patient includes examination data from an X-ray, MRT and/or ultrasound examination.

11. The set as claimed in claim 8 further comprising an orientation aid on which markings are placed which define a two-dimensional coordinate grid, wherein the orientation aid comprises a fastening surface which is configured to fasten the orientation aid to the patient.

12. The set as claimed in claim 11, wherein the orientation aid is a flat flexible element.

13. The set as claimed in claim 11 wherein the examination data of the patient includes positional information relating to the orientation aid fastened to the patient, and wherein the computer program is set up to calculate an output of the position specifications and/or of the angular orientation specifications as a function of the positional information relating to the orientation aid on the patient.

14. The set as claimed in claim 11 wherein the orientation aid has a plurality of marker elements which are automatically detectable using X-ray, MRT and/or ultrasound images.

15. A set comprising a plurality of elements for carrying out an examination and/or treatment of a patient, comprising:
a) an orientation aid on which markings are placed which define a two-dimensional coordinate grid, wherein the orientation aid has a fastening surface which is configured to fasten the orientation aid to the patient,
b) a computer program with instructions encoded on a non-transient medium which are executable on a computer, the computer program (101) being set up to use
characteristic data of the orientation aid,
examination data of the patient, and
at least one position on the patient selectable from the examination data of the patient,
in order to calculate coordinate specifications in the two-dimensional coordinate grid of the orientation aid, and output said coordinate specifications when the computer program is run on the computer.

16. A computer program with instructions encoded on a non-transient medium which are executable on a computer, the computer program being set up to use
characteristic data of a needle-guiding device,
examination data of the patient, and
at least one predetermined examination and/or treatment step to be carried out with the needle-guiding device
in order to calculate
b1) positioning specifications for positioning of the needle-guiding device on the patient
and/or
b2) one or more angular orientation specifications for setting an angular orientation of a needle-guiding element relative to a base element (7) in at least one degree of angular freedom or in several mutually independent degrees of angular freedom
and to output these one or more angular orientation specifications when the computer program is run on the computer.

17. A computer program with instructions encoded on a non-transient medium which are executable on a computer, the computer program being set up to use
characteristic data of an orientation aid,
examination data of the patient, and
at least one position on the patient, selected from the examination data of the patient,
in order to calculate coordinate specifications in a coordinate grid of the orientation aid and output said coordinate specifications when the computer program is run on the computer.
